(19) United States
(12) Patent Application Publication
(10) Pub. No.: US 2023/0366795 A1
(43) Pub. Date: Nov. 16, 2023

(54) APPARATUS FOR SUPPORTING A TISSUE SPECIMEN

(71) Applicant: CytoVeris Inc., Farmington, CT (US)

(72) Inventors: Morgan McNamara, Danbury, CT (US); David Fournier, Northborough, MA (US); Noel Diagneault, Burlington, CT (US); Michael A. Sapack, Southbury, CT (US); Gary Root, Cheshire, CT (US)

(21) Appl. No.: 18/196,411

(22) Filed: May 11, 2023

Related U.S. Application Data
(60) Provisional application No. 63/340,758, filed on May 11, 2022.

(51) Int. Cl. G01N 1/36; A61B 90/00; A61B 90/90

(52) U.S. Cl. CPC G01N 1/36; A61B 90/39; A61B 90/90; A61B 2090/3937; B01L 2200/141; B01L 2300/123; B01L 2300/041; B01L 2300/021; B01L 2300/12

(57) ABSTRACT

An apparatus for holding a tissue specimen during imaging is provided. The tissue specimen has a 3D geometry and a region of interest. The apparatus includes a cover panel and a specimen support structure. The cover panel has upper and bottom panel surfaces and a viewing aperture that extends between the upper and bottom panel surfaces. The specimen support structure is configured to support the tissue specimen in an imaging orientation. The specimen support structure is configured to permit alignment of the tissue specimen ROI with the viewing aperture. The apparatus is configurable in a secured configuration in which the specimen support structure is positionally fixed relative to the cover panel, the tissue specimen ROI is exposed through the viewing aperture for imaging, and the tissue specimen ROI is held motionless relative to the cover panel.

This application claims priority to U.S. Patent Appln. No. 63/340,758 filed May 11, 2022, which is hereby incorporated by reference in its entirety.

### BACKGROUND OF THE INVENTION

### 1. Technical Field

The present disclosure relates to structures for mounting tissue samples in general, and to structures for mounting tissue samples for imaging in particular.

### 2. Background Information

It is important to keep a tissue specimen still when capturing images of the tissue specimen for subsequent analysis. This is particularly true when the analysis requires multiple images of the same specimen. Tissue specimen movement can produce inconsistencies (e.g., registration errors) between the multiple images that in turn can negatively affect the subsequent analysis, or at the very least require corrective action. In addition, unintended tissue specimen movement can result in contact between the tissue specimen and optical components, thereby potentially causing contamination of the contacted optical components.

What is needed is a system or apparatus that facilitates keeping a tissue specimen still during imaging.

### SUMMARY

According to an aspect of the present disclosure, an apparatus for holding a tissue specimen during imaging is provided. The tissue specimen has a three-dimensional geometry and a region of interest (ROI). The apparatus includes a cover panel and a specimen support structure. The cover panel has an upper panel surface, a bottom panel surface opposite the upper panel surface, and a viewing aperture that extends between the upper panel surface and the bottom panel surface. The specimen support structure is configured to support the tissue specimen in an imaging orientation. The specimen support structure is configured to permit alignment of the tissue specimen ROI with the viewing aperture. The apparatus is configurable in a secured configuration. In the secured configuration the specimen support structure is positionally fixed relative to the cover panel, the tissue specimen ROI is exposed through the viewing aperture for imaging, and the tissue specimen ROI is held motionless relative to the cover panel.

In any of the aspects or embodiments described above and herein, the cover panel may be configured to be photometrically benign.

In any of the aspects or embodiments described above and herein, the cover panel may be configured to not fluoresce.

In any of the aspects or embodiments described above and herein, the cover panel may be photometrically non-transmissive.

In any of the aspects or embodiments described above and herein, in the secured configuration the specimen support structure and the cover panel may be biased toward one another.

In any of the aspects or embodiments described above and herein, the viewing aperture may be configured to isolate the tissue specimen ROI when the apparatus is disposed in the secured configuration.

In any of the aspects or embodiments described above and herein, the cover panel may include one or more data markings disposed on the upper panel surface.

In any of the aspects or embodiments described above and herein, the data markings may be fiducial markings.

In any of the aspects or embodiments described above and herein, the data markings may be configured to provide patient information.

In any of the aspects or embodiments described above and herein, the apparatus may further include a base plate, and the specimen support structure may be disposed on the base plate and be movable relative to the base plate and the cover panel when the apparatus is in an unsecured configuration. The cover plate may be attached to the base plate when the apparatus is in the secured configuration.

In any of the aspects or embodiments described above and herein, in the secured configuration the base plate and the cover panel may be biased toward one another.

In any of the aspects or embodiments described above and herein, the specimen support structure may include a pedestal structure disposed within an open container.

In any of the aspects or embodiments described above and herein, the specimen support structure may include an array of individual support elements extending outwardly from a base panel, and the individual support elements may be movable relative to one another to define a geometry that mates with at least a portion of the tissue specimen.

In any of the aspects or embodiments described above and herein, the specimen support structure may include an array of individual support elements and a base panel having an array of base panel apertures. The individual support elements may be each configured to engage with a respective base panel aperture in a manner such that the respective support element extends outwardly from the at least one base panel. At least one of the plurality of individual support elements may have a first geometric configuration, and at least another of the plurality of individual support elements may have a second geometric configuration which is different from the first geometric configuration.

In any of the aspects or embodiments described above and herein, the specimen support structure may include a container containing a magnetic material, and the apparatus may include a controllable magnetic field source. The container containing the magnetic material is disposable in a geometry that mates with at least a portion of the tissue specimen, and the magnetic field source is controllable to selectively produce a magnetic field that maintains the magnetic material in the geometry.

In any of the aspects or embodiments described above and herein, the specimen support structure may include a container containing a non-Newtonian material.

In any of the aspects or embodiments described above and herein, the specimen support structure may include a deformable panel that is geometrically deformable to define a geometry that mates with at least a portion of the tissue specimen.

In any of the aspects or embodiments described above and herein, the specimen support structure may include a plurality of individual support elements and a base panel having an array of channels. Each individual support element may be configured to engage with a respective channel.

In any of the aspects or embodiments described above and herein, the specimen support structure may include a tapered interior cavity.

The foregoing features and elements may be combined in various combinations without exclusivity, unless expressly indicated otherwise. For example, aspects and/or embodiments of the present disclosure may include any one or more of the individual features or elements disclosed above and/or below alone or in any combination thereof. These features and elements as well as the operation thereof will become more apparent in light of the following description and the accompanying drawings. It should be understood, however, the following description and drawings are intended to be exemplary in nature and non-limiting.

### DESCRIPTION OF THE DRAWINGS

FIG. 1 is a diagrammatic view of a tissue specimen imaging/analysis system embodiment.

FIG. 2 is a diagrammatic view of a tissue specimen illustrating Z-axis imaging planes.

FIG. 3 is a planar view and an end view of a present disclosure cover panel embodiment.

FIG. 4 is a diagrammatic view of a present disclosure holder embodiment including a specimen support structure embodiment.

FIG. 5 is a diagrammatic view of a present disclosure tissue specimen holder embodiment with a tissue specimen and without a tissue specimen.

FIG. 6 is a diagrammatic view of a present disclosure holder embodiment including a specimen support structure embodiment.

FIGS. 7 and 7A are diagrammatic views of a present disclosure tissue specimen holder embodiment.

FIGS. 8-14 are diagrammatic views of present disclosure tissue specimen holder embodiments.

FIG. 15 is a diagrammatic view of a cover panel with an attached contamination control skirt.

### DETAILED DESCRIPTION

Embodiments of the present disclosure provide a novel and unobvious apparatus (e.g., a specimen holder) for holding a tissue specimen 20 in a manner that facilitates imaging. The present disclosure is directed to holding a variety of different types of excised tissue specimens 20, and therefore is not limited to any particular type of tissue specimen 20. A non-limiting example of a tissue specimen 20 with which the present disclosure may be used is an excised prostate tissue specimen; e.g., a prostate tissue specimen excised during a robotic assisted radical prostatectomy (RARP) procedure. The term “imaging” as used herein refers to any photometric process in which light emanating from the tissue specimen 20 is captured/detected in a form that may be subsequently used for analysis of the tissue specimen 20. A non-limiting example of a system that utilizes imaging for analysis of a tissue specimen 20 is disclosed in U.S. patent application Ser. No. 18/027,022 “Multi-Spectral Imager for UV-Excited Tissue Autofluorescence Mapping”, which is hereby incorporated by reference in its entirety. FIG. 1 diagrammatically illustrates an example of such a system. The system utilizes one or more light sources to produce excitation light at one or more wavelengths incident to a tissue specimen 20 (and more specifically a region of interest 22 or “ROI 22” of the tissue specimen 20) and utilizes one or more light detectors (e.g., a camera) to capture autofluoresence (AF) emitted from endogenous biomolecules within the tissue specimen 20 and/or light reflected from endogenous biomolecules within the tissue specimen 20. The system processes the captured light to analyze the tissue specimen 20. The present disclosure is not limited to use with any particular tissue specimen imaging system and U.S. patent application Ser. No. 18/027,022 is referenced here as an example.

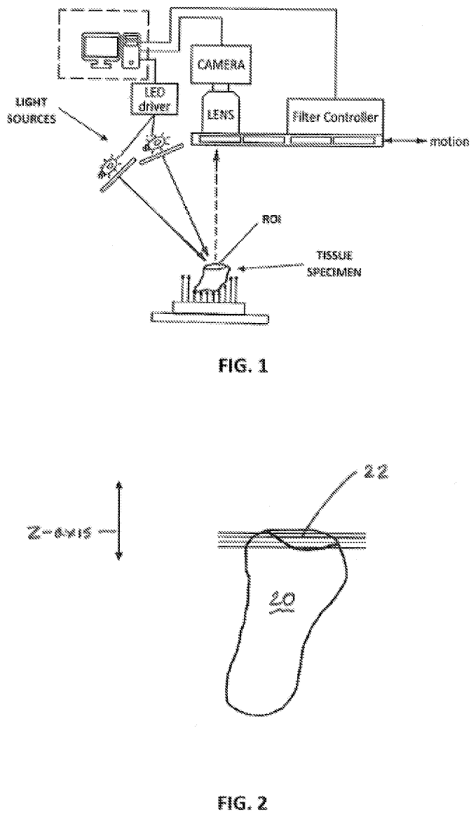

Endogenous emitted AF (sometimes referred to as a “fluorescent signature”) may offer useful information that can be mapped to the functional, metabolic and morphological attributes of the tissue, and therefore may be utilized for diagnostic purposes [1-5]. The biomolecules present in the tissue provide discernible and repeatable AF spectral patterns [6] that can be interpreted as a “biomolecular optical fingerprint”. A system like that disclosed in U.S. patent application Ser. No. 18/027,022 may acquire several images of a tissue specimen 20 through different focus planes and with different excitation and emission/reflectance wavelengths of a three dimensional surface of the specimen 20. The system may acquire a panel of multispectral images for use in the analysis. For example, when a tissue specimen 20 has a three-dimensional geometric configuration, the imaging process may include acquiring multiple images along a Z-axis that extends toward and away from the imaging system objective lens. FIG. 2 diagrammatically illustrates a tissue specimen 20 having a ROI 22, and multiple Z-axis planes for imaging. The multiple Z-axis images (each two-dimensional) may be stacked to construct a focused three-dimensional image of the tissue specimen surface. To account for tissue morphological or textural attributes during tissue analysis/classification, all the multispectral images must preserve spatial location. In some instances, the system may use Artificial Intelligence (AI) and Machine Learning (ML) to analyze an imaged tissue specimen 20.

Systems like that disclosed in U.S. patent application Ser. No. 18/027,022 typically include a stage configured to support a tissue specimen 20 and an imaging system having an objective lens. The stage may be movable relative to the objective lens, or the objective lens may be movable relative to the stage, or both may be movable relative to the other. In these systems, the relative movement between the stage and the objective lens is typically purposeful and defined; e.g., for adjusting focal length, to image different portions of the tissue specimen 20 and the like, or any combination thereof.

It is often the case that a particular region of a tissue specimen 20 is of interest (i.e., an ROI 22) and it is important to appropriately orient the aforesaid ROI 22 relative to the objective lens for imaging purposes and to maintain that orientation without movement to permit multiple images of exactly the same ROI 22. The present disclosure specimen holder embodiments are configured to allow a user to dispose and maintain a specimen 20 in a desired orientation for imaging and to physically and photometrically isolate the ROI 22 to facilitate the imaging.

The present disclosure holder 24 includes a specimen support structure 26 and an apertured cover panel 28 that cooperate with one another. The specimen support structure 26 allows the user to orient the specimen 20 for imaging and supports the specimen 20 in that orientation and the apertured cover panel 28 physically and photometrically isolates an ROI 22.

FIG. 3 diagrammatically illustrates an apertured cover panel 28. The cover panel 28 is typically a planar body having an upper panel surface 30, an opposite bottom panel surface 32, and a viewing aperture 34 that extends between the upper and bottom panel surfaces 30, 32. The viewing aperture 34 is not limited to any particular area or geometry. In some applications, a plurality of cover panels 28 are available and a cover panel 28 having a viewing aperture 34 that is sized appropriately for the ROI 22 under consideration is chosen. The cover panel 28 is sufficiently rigid to maintain a planar configuration during uses such as those described herein. The cover panel 28 may be comprised of different materials for different imaging applications, and is typically photometrically benign; i.e., during the imaging process the cover panel 28 typically does not produce a photometric response (e.g., fluorescence) that may confound the imaging process. In some instances, the cover panel 28 may include a coating that makes the cover panel 28 photometrically benign. In some instances, the cover panel 28 may be configured to be photometrically non-transmissive to ensure the ROI 22 that is exposed in the viewing aperture 34 is photometrically isolated from the remainder of the tissue specimen 20.

In some embodiments (as shown in FIG. 3), information, or interpretable markings that can be used for specimen 20 identification, patient identification, analysis identification (e.g., time, date, testing entity, or the like), or fiducial markings, or an image reference block operable to facilitate imaging (e.g., an optical reference material configured to provide an optical measurement reference such as reflectance, or fluorescence, or the like), or any combination thereof (all generically referred herein as a “data marking”—shown in FIG. 3 with reference number 36) may be disposed on the upper panel surface 30 of the cover panel 28. These markings 36 may be interpretable by human eye (e.g., written text) or may be machine interpretable (e.g., bar codes, image reference blocks, and the like), or any combination thereof. These data markings 36 are understood to facilitate imaging and/or imaging data identification/custody; e.g., imaging applied, date, patient name, facility, and the like.

The specimen support structure 26 may assume a variety of different configurations. Non-limiting examples of support structures 26 are described below.

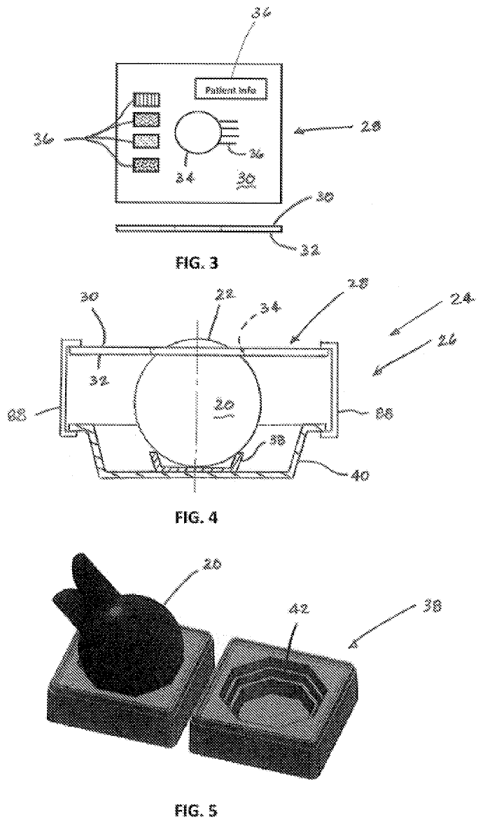

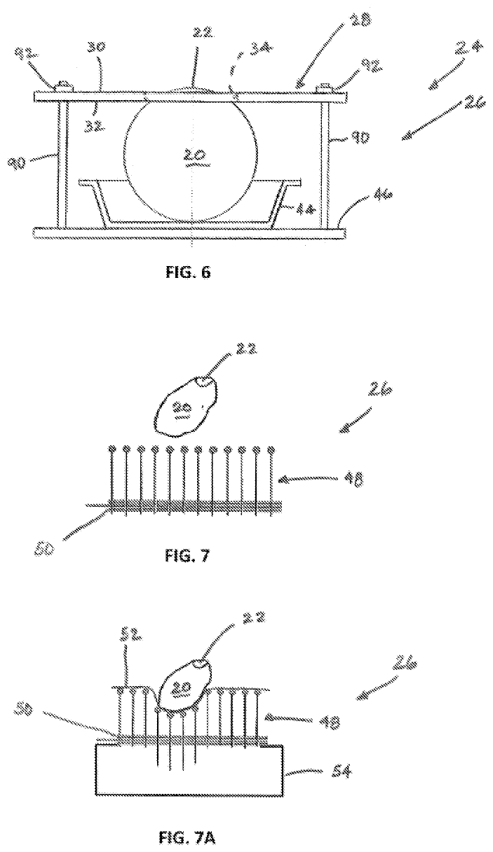

FIG. 4 diagrammatically illustrates a specimen support structure 26 example that includes a first container 38 (e.g., a weigh boat) sized to receive a portion of the tissue specimen 20. The first container 38 is disposed within a second container 40 that is preferably configured larger than the tissue specimen 20 and is configured to hold a liquid. In this manner, any liquid or other debris from the tissue specimen 20 can be caught and retained to avoid spillage that may contaminate the imaging system. The first container 38 acts as a pedestal for the tissue specimen 20 within the second container 40. The first container 38 is not limited to any particular geometry. FIG. 5 illustrates an example of a first container 38 that includes a stepped interior cavity 42 with the size of the interior cavity 42 increasing with each step. FIG. 5 illustrates octagonal-shaped steps shown but the steps may be otherwise configured; e.g., rectangular, circular, elliptical, and the like. The first container 38 is not fixed within the second container 40 and therefore permits the tissue specimen 20 to be moved (e.g., translated in either of two axes of movement—X-axis or Y-axis, and/or rotated) relative to the second container 40. In this manner, the tissue specimen 20 may be readily manipulated to position the ROI 22 in a desired orientation. FIG. 6 illustrates another embodiment wherein the specimen 20 is disposed in a container 44 for selective positioning. In this embodiment, the specimen support structure 26 includes a base plate 46 and the container 44 is disposed on top of the base plate 46. The container 44 is not fixed relative to the base plate 46 and therefore the container 44 (and tissue specimen 20) can be moved (e.g., translated in either of two axes of movement—X-axis or Y-axis, and/or rotated) relative to the base plate 46 to position the ROI 22 in a desired orientation.

FIGS. 7 and 7A diagrammatically illustrate another example of a specimen support structure 26 that includes an array of displaceable linear support elements 48 extending from a base panel 50. The linear support elements 48 can be moved vertically relative to the base panel 48; e.g., certain of the linear support elements 48 can be displaced to produce a “pocket” having a geometry that produces the desired the tissue specimen 20 orientation. FIG. 7 illustrates the linear support elements 48 prior to a pocket being formed and FIG. 7A illustrates the linear support elements 48 translated to form a pocket, and a tissue specimen 20 disposed in the pocket. The base panel 50 is configured to allow translation of the linear support elements 48 for pocket creation and thereafter to hold the linear support elements 48 in their respective pocket positions. FIG. 7A illustrates a flexible cover panel 52 (e.g., a “protective layer”) that serves as an interface between the linear support elements 48 and the tissue specimen 20 (e.g. to prevent contamination of the specimen support structure 26) and a riser 54 that accepts portions of the linear support elements 48 and maintains the position of the base panel 50. The linear support elements 48 and the base panel 50 support the specimen 20 and enable the user to disposed the specimen 20 in a desired orientation.

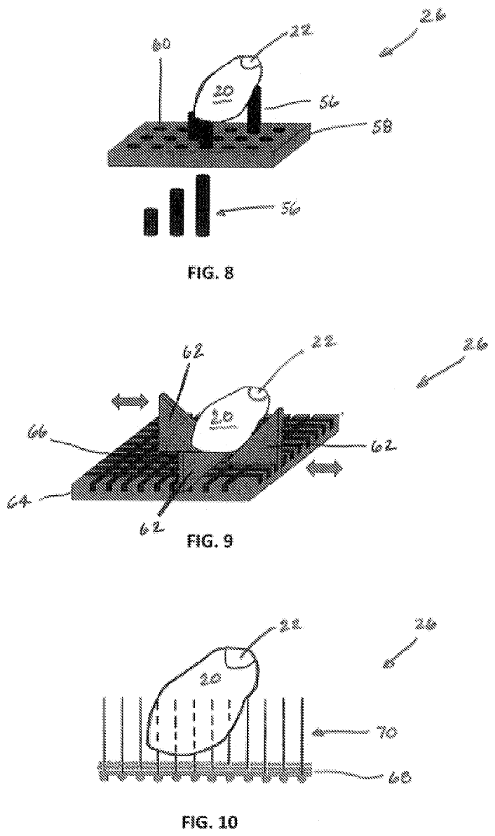

FIG. 8 diagrammatically illustrate another example of a specimen support structure 26 that includes a plurality of linear support elements 56 engageable with a base panel 58. In this embodiment, the base panel 58 includes an array of apertures 60 and the linear support elements 56 are provided in a plurality of different heights. FIG. 8 diagrammatically illustrates three linear support elements 56, each having a different height, that may be engaged with the base panel 58 to hold the tissue specimen 20 in the desired orientation. The linear support elements 56 may be insertable within the apertures 60 as a slide fit or as a slight interference fit, or the linear support elements 56 and the apertures 60 may have a threaded engagement, or the like. The linear support elements 56 and the base panel 58 support the specimen 20 and enable the user to disposed the specimen 20 in a desired orientation.

FIG. 9 diagrammatically illustrate another example of a specimen support structure 26 that includes a plurality of support elements 62 engageable with a base panel 64. In this embodiment, the base panel 64 includes an array of orthogonal channels 66 configured to receive an edge of a support element 62. The support elements 62 are shown in FIG. 9 as being generally triangular, but the present disclosure is not limited to any particular support element 62 geometry. In fact, the present disclosure may utilize a variety of different support element 62 geometries to facilitate holding the tissue specimen 20. The present disclosure is also not limited to a base panel 64 having orthogonal channels 66; e.g., the base panel 64 and support elements 62 may be configured for other physical connection like Lego® cylindrical protrusions that can be disposed in cylindrical apertures. The support elements 62 and the base panel 64 support the specimen 20 and enable the user to disposed the specimen 20 in a desired orientation.

FIG. 10 diagrammatically illustrate another example of a specimen support structure 26 that includes a base panel 68 and a plurality of support elements 70 extending outwardly from the base panel 68; e.g., a horizontally oriented base panel 68 and vertically oriented support elements 70. The support elements 70 are configured (e.g., pin or needle-like structures) to pierce the tissue specimen 20 when the specimen 20 is pressed against the support elements 70. Friction between the support elements and the tissue specimen 20 thereafter hold the specimen 20 in position. The support elements 70 and the base panel 68 support the specimen 20 and enable the user to disposed the specimen 20 in a desired orientation.

FIG. 11 diagrammatically illustrate another example of a specimen support structure 26 that includes magnetic material 72 disposed in a container 74. In this example, a magnetic field source is included that can be switched between an “OFF” configuration where no magnetic field is produced and an “ON” configuration where a magnetic field is produced. The magnetic material 72 may be beads (e.g., as shown in FIG. 11), or a magneto-rheological fluid, or the like. In the absence of an applied magnetic field, the magnetic material 72 may be displaced to assume a geometry that positions and maintains the tissue specimen 20 in a particular orientation. Once the desired magnetic material 72 geometry is established, the magnetic field source may be switched to an “ON” configuration to produce a magnetic field and the magnetic field will cause the magnetic material 72 to maintain the geometry that positions and maintains the tissue specimen 20 in the desired orientation.

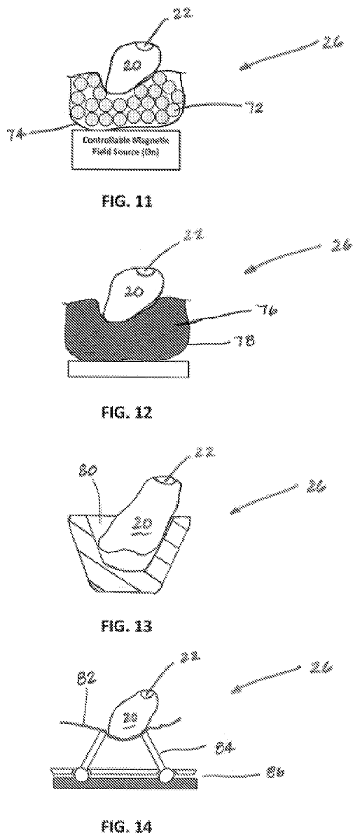

FIG. 12 diagrammatically illustrate another example of a specimen support structure 26 that includes a non-Newtonian fluid 76 disposed in a container 78. The non-Newtonian fluid 76 can be deformed and thereafter will maintain its geometry for an acceptable period of time. Alternatively, the container 78 may contain a material (e.g., a granular material) that can be deformed and friction within the material will maintain the geometry of the material for an acceptable period of time. The container 78 containing the non-Newtonian fluid or frictional granular material enables the user to dispose the specimen 20 in a desired orientation.

FIG. 13 diagrammatically illustrate another example of a specimen support structure 26 that includes a tapered (e.g., conical) interior cavity 80 configured to receive and hold the tissue specimen 20. The taper angle of the surfaces defining the interior cavity 80 can be varied to accommodate different tissue specimen 20 configurations; e.g., a first embodiment with a tapered interior cavity 80 disposed at first angles, a second embodiment with a tapered interior cavity 80 disposed at second angles different from the first angles, and so on.

FIG. 14 diagrammatically illustrate another example of a specimen support structure 26 that includes a deformable panel 82 (e.g., a solid panel, an apertured panel, a net, or the like) that can be configured to support the specimen 20. The deformable panel 82 may be supported by a plurality of support elements 84 (e.g., rods, pillars, or the like) engageable with the base panel 86.

Any of the specimen support structures 26 described herein may include a flexible cover layer/protective layer 52 (e.g., a sheet of material such as plastic, or foil, or the like as shown in FIG. 7A) disposed between the specimen support structure 26 and the tissue specimen 20 as a barrier to maintain cleanliness of the specimen support structure 26, to prevent contamination between the tissue specimen 20 and the specimen support structure 26, or for any other purpose useful to the specimen imaging/analysis process.

As indicated above, the present disclosure holder 24 includes a specimen support structure 26 and an apertured cover panel 28 that cooperate with one another to support and orient the specimen 20 for imaging and to physically and photometrically isolate an ROI 22. In the holder 24 embodiment diagrammatically shown in FIG. 4, the tissue specimen 20 is disposed in the first container 38 and the first container 38, in turn, is disposed in the second container 40. The first container 38 functions as a pedestal to support the specimen 20 and allow the specimen 20 to be moved relative to the second container 40 and the cover panel 28 to align an ROI 22 of the specimen 20 with the viewing aperture 34 of the cover panel 28; i.e., an unsecured configuration. Once alignment is achieved, the cover panel 28 and second container 40 may be secured to one another; i.e., a secured configuration. FIG. 4 diagrammatically illustrates clamps 88 that apply a biasing force urging the cover panel 28 toward the second container, and vice versa. The clamps 88 are a non-limiting example of a mechanical structure that may be used to engage the aspects of the support structure 26 (i.e., the second container 40) and the cover panel 28. The present disclosure is not limited to using clamps 88. Once the cover panel 28 and second container 40 are secured, the viewing aperture 34 is physically engaged with the specimen 20 in a manner that prevents the ROI 22 from moving relative to the viewing aperture 34; i.e., the ROI 22 may be described as being physically isolated. Also as indicated herein, the cover panel 28 may be configured (e.g., by material choice, by coating or the like) to be photometrically benign and/or non-transmissive. In the secured configuration, the cover panel 28 isolating the ROI 22 therefore also may photometrically isolate the ROI 22 from the remainder of the tissue specimen 20. In the secured configuration, the holder 24 may be placed on the stage of an imaging system like that identified herein for analysis with the ROI 22 correctly oriented and in a non-movable state that facilitates imaging.

In the holder 24 embodiment diagrammatically shown in FIG. 6, the tissue specimen 20 is disposed in the container 44 and the container 44, in turn, is disposed on the base plate 46. The container 44 supports the specimen 20 and allows the specimen 20 to be moved relative to the cover panel 28 to align an ROI 22 of the specimen 20 with the viewing aperture 34 of the cover panel 28. Once alignment is achieved, the base plate 46 and the cover panel 28 may be secured to one another. FIG. 6 diagrammatically illustrates posts 90 that include a fastener 92 that can be actuated to apply a biasing force urging the cover panel 28 toward the base plate 46, and vice versa. The posts 90 and fasteners 92 are a non-limiting example of a mechanical structure that may be used to engage the aspects of the specimen support structure 26 (i.e., the base plate) and the cover panel 28. The present disclosure is not limited to using posts 90 and fasteners 92. Once the cover panel 28 and base plate 46 are secured, the viewing aperture 34 is physically engaged with the specimen 20 in a manner that prevents the ROI 22 from moving relative to the viewing aperture 34; i.e., the ROI 22 may be described as being physically isolated and the cover panel 28 may photometrically isolate the ROI 22 from the remainder of the tissue specimen 20 as detailed herein. In the secured configuration, the holder 24 may be placed on the stage of an imaging system like that identified herein for analysis with the ROI 22 correctly oriented and in a non-movable state that facilitates imaging.

The holder 24 embodiments diagrammatically illustrated in FIGS. 4 and 6 are non-limiting examples of how a specimen support structure 26 and an apertured cover panel 28 may cooperate with one another to support and orient the specimen 20 for imaging and to physically and photometrically isolate an ROI 22. The present disclosure is not limited to these examples. For example, the specimen support structure 26 embodiments detailed herein and shown in FIGS. 7-14 may be used in a holder 24 arrangement like that diagrammatically shown in FIG. 4; e.g., any of the specimen support structure 26 embodiments detailed herein and shown in FIGS. 7-14 may be disposed in the second container 40 shown in FIG. 4 in place of the first container 38. As another example, the specimen support structure 26 embodiments detailed herein and shown in FIGS. 7-14 may be used in a holder 24 arrangement like that diagrammatically shown in FIG. 6; e.g., any of the specimen support structure 26 embodiments detailed herein and shown in FIGS. 7-14 may be disposed on the base plate 46 in place of the container 44.

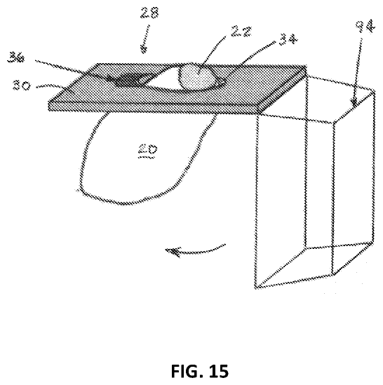

In some embodiments, the present disclosure holder 24 may include a contamination control skirt configured to avoid the tissue specimen 20 contaminating the system or components within the system. Non-limiting examples of contamination control skirt configurations include a planar material, or a plastic bag, or the like that is disposed around a portion or all of the periphery of the holder 24. FIG. 15 diagrammatically illustrates a contamination control skirt 94 attached to one edge of a cover panel 28. The contamination skirt 94 may be liquid impermeable. The contamination control skirt 94 can be rotated clockwise and attached elsewhere to the cover panel 28 to enclose the specimen 20 to minimize the possibility of contamination.

While the principles of the disclosure have been described above in connection with specific apparatuses and methods, it is to be clearly understood that this description is made only by way of example and not as limitation on the scope of the disclosure. Specific details are given in the above description to provide a thorough understanding of the embodiments. However, it is understood that the embodiments may be practiced without these specific details.

It is noted that the embodiments may be described as a process which is depicted as a flowchart, a flow diagram, a block diagram, etc. Although any one of these structures may describe the operations as a sequential process, many of the operations can be performed in parallel or concurrently. In addition, the order of the operations may be rearranged. A process may correspond to a method, a function, a procedure, a subroutine, a subprogram, etc.

The singular forms “a,” “an,” and “the” refer to one or more than one, unless the context clearly dictates otherwise. For example, the term “comprising a specimen” includes single or plural specimens and is considered equivalent to the phrase “comprising at least one specimen.” The term “or” refers to a single element of stated alternative elements or a combination of two or more elements unless the context clearly indicates otherwise. As used herein, “comprises” means “includes.” Thus, “comprising A or B,” means “including A or B, or A and B,” without excluding additional elements.

It is noted that various connections are set forth between elements in the present description and drawings (the contents of which are included in this disclosure by way of reference). It is noted that these connections are general and, unless specified otherwise, may be direct or indirect and that this specification is not intended to be limiting in this respect. Any reference to attached, fixed, connected or the like may include permanent, removable, temporary, partial, full and/or any other possible attachment option.

No element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprise”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures—such as alternative materials, structures, configurations, methods, devices, and components, and so on—may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein. For example, in the exemplary embodiments described above within the Detailed Description portion of the present specification, elements may be described as individual units and shown as independent of one another to facilitate the description. In alternative embodiments, such elements may be configured as combined elements. It is further noted that various method or process steps for embodiments of the present disclosure are described herein. The description may present method and/or process steps as a particular sequence. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the description should not be construed as a limitation.

### REFERENCES

1. R. A. Schwarz et al., Autofluorescence and diffuse reflectance spectroscopy of oral epithelial tissue using a depth-sensitive fiber-optic probe. Appl Opt 47, 825-834 (2008).
2. W. Zheng et al., Optimal excitation-emission wavelengths for autofluorescence diagnosis of bladder tumors. Int J Cancer 104, 477-481 (2003).
3. T. A. Valdez et al., Multiwavelength fluorescence otoscope for video-rate chemical imaging of middle ear pathology. Anal Chem 86, 10454-10460 (2014).
4. R. Pandey et al., AI-powered biomolecular-specific and label-free multi-spectral imaging rapidly detects malignant neoplasm in surgically excised breast tissue specimens”, Archives of Pathology & Laboratory Medicine, 2022.
5. T. M. Bydlon et al., Chromophore based analyses of steady-state diffuse reflectance spectroscopy: current status and perspectives for clinical adoption. J Biophotonics 8, 9-24 (2015).
6. A. C. Croce et al., Autofluorescence spectroscopy and imaging: a tool for biomedical research and diagnosis. Eur J Histochem. 2014; 58(4):2461.

1. An apparatus for holding a tissue specimen during imaging, the tissue specimen having a three-dimensional geometry and a region of interest (ROI), the apparatus comprising:
a cover panel having an upper panel surface, a bottom panel surface opposite the upper panel surface, and a viewing aperture extending between the upper panel surface and the bottom panel surface; and
a specimen support structure configured to support the tissue specimen in an imaging orientation;
wherein the specimen support structure is configured to permit alignment of the tissue specimen ROI with the viewing aperture; and
wherein the apparatus is configurable in a secured configuration, and in the secured configuration the specimen support structure is positionally fixed relative to the cover panel and the tissue specimen ROI is exposed through the viewing aperture for imaging and the tissue specimen ROI is held motionless relative to the cover panel.

2. The apparatus of claim 1, wherein the cover panel is configured to be photometrically benign.

3. The apparatus of claim 2, wherein the cover panel is configured to not fluoresce.

4. The apparatus of claim 2, wherein the cover panel is photometrically non-transmissive.

5. The apparatus of claim 1, wherein in the secured configuration the specimen support structure and the cover panel are biased toward one another.

6. The apparatus of claim 1, wherein the viewing aperture is configured to isolate the tissue specimen ROI when the apparatus is disposed in the secured configuration.

7. The apparatus of claim 1, wherein the cover panel includes one or more data markings disposed on the upper panel surface.

8. The apparatus of claim 7, wherein the data markings are fiducial markings.

9. The apparatus of claim 7, wherein the data markings are configured to provide patient information.

10. The apparatus of claim 7, wherein the data markings is an optical reference material configured to provide an optical measurement reference.

11. The apparatus of claim 1, further comprises a base plate; and
wherein the specimen support structure is disposed on the base plate and is movable relative to the base plate and the cover panel when the apparatus is in an unsecured configuration;
wherein the cover plate is attached to the base plate when the apparatus is in the secured configuration.

12. The apparatus of claim 11, wherein in the secured configuration the base plate and the cover panel are biased toward one another.

13. The apparatus of claim 1, wherein the specimen support structure includes a pedestal structure disposed within an open container.

14. The apparatus of claim 1, wherein the specimen support structure includes an array of individual support elements extending outwardly from a base panel; and
wherein the individual support elements are movable relative to one another to define a geometry that mates with at least a portion of the tissue specimen.

15. The apparatus of claim 1, wherein the specimen support structure includes an array of individual support elements and a base panel having an array of base panel apertures; and
wherein the individual support elements are each configured to engage with a respective said base panel aperture in a manner such that the respective support element extends outwardly from the at least one base panel; and
wherein at least one of the plurality of individual support elements has a first geometric configuration, and at least another of the plurality of individual support elements has a second geometric configuration which is different from the first geometric configuration.

16. The apparatus of claim 1, wherein the specimen support structure includes a container containing a magnetic material; and
the apparatus includes a controllable magnetic field source;
wherein the container containing the magnetic material is disposable in a geometry that mates with at least a portion of the tissue specimen, and the magnetic field source is controllable to selectively produce a magnetic field that maintains the magnetic material in the geometry.

17. The apparatus of claim 1, wherein the specimen support structure includes a container containing a non-Newtonian material.

18. The apparatus of claim 1, wherein the specimen support structure includes a deformable panel that is geometrically deformable to define a geometry that mates with at least a portion of the tissue specimen.

19. The apparatus of claim 1, wherein the specimen support structure includes a plurality of individual support elements and a base panel having an array of channels, and each individual support element is configured to engage with a said channel.

20. The apparatus of claim 1, wherein the specimen support structure includes a tapered interior cavity.

21. The apparatus of claim 1, wherein the cover panel includes a contamination control skirt attached to the cover panel that is liquid impermeable and configured to enclose the tissue specimen.
